(19) United States
(12) Patent Application Publication
(10) Pub. No.: US 2014/0228552 A1
(43) Pub. Date: Aug. 14, 2014

(54) MRI CONTRAST ENHANCING AGENT

(71) Applicant: NATIONAL CHENG KUNG UNIVERSITY, Tainan City (TW)

(72) Inventor: Ping-Ching WU, Tainan City (TW)

(73) Assignee: National Cheng Kung University, Tainan City (TW)

(21) Appl. No.: 13/762,580

(22) Filed: Feb. 8, 2013

(51) Int. Cl. A61K 49/08

(52) U.S. Cl. CPC A61K 49/085
USPC 534/16

(57) ABSTRACT

The present invention provides a MRI contrast enhancing agent, which comprises: a magnetic particle with a T2-reducing effect; a polymer coupled to a surface of the magnetic particle; and a T1 contrast agent coupled to the polymer.

### BACKGROUND OF THE INVENTION

1. Field of the Invention

The present invention relates to a MRI contrast enhancing agent and, more particularly, to a MRI negative contrast enhancing agent with enhanced T1-reducing effect.

2. Description of Related Art

Imaging examinations are general clinical methods used in diagnostics and therapeutics. During performing the imaging examinations, reducing agents are generally used to enhance the contrast between different tissues, in order to improve the visibility of tissues to be examined. In recent years, one common imaging examinations used in clinic is magnetic resonance imaging (MRI).

MRI is a medical imaging method to visualize detailed internal structures of an object. During the examination, the MRI machine provides a powerful magnetic field to align the magnetization of hydrogen atoms in the body, and then a section image of an organ of the object can be obtained after digital processing. The MRI technique has advantages of non-ionizing radiation, and high sensitivity to the distribution of water and other biomolecules, so it has been applied in clinic for the diagnosis of many diseases. Furthermore, MRI has good contrast between different soft tissues of the body, so it does not need any imaging contract agents for most of the MRI examination. However, some reducing agents still have to be applied to the body when fine MRI examination is to be performed.

Clinically used MRI contrast agents can be divided into T1 contrast agents such as gadolinium (Gd³⁺)-based contrast agents, and T2 contrast agents such as iron oxide-based contrast agents. However, in order to obtain desired MRI enhancement effect, large doses of these commercial available MRI contrast agents have to be applied to subjects, but such large doses of MRI contrast agents may be physiologically intolerable to the subjects. Hence, it is desirable to provide a novel MRI contrast agents with enhancement contrast effect, in order to reduce the administration amount of the MRI contrast agents to patients.

### SUMMARY OF THE INVENTION

The object of the present invention is to provide a MRI contrast enhancing agent, which has improved T1-reducing effect as well as T2-reducing effect.

To achieve the object, the MRI contrast enhancing agent comprises: a magnetic particle with a T2-reducing effect; a polymer coupled to a surface of the magnetic particle; and a T1 contrast agent coupled to the polymer. In the present invention, the term “T2” does not mean “T2” only, but also comprises the meaning of “T2*” as well.

The MRI contrast enhancing agent of the present invention can be used as a MRI negative contrast agent. The inventor found that when the magnetic particle with the T2-reducing effect is coupled with the T1 contrast agent, it can not only be used as a carrier for the T1 contrast agent but also enhance the T1-reducing effect of the T1 contrast agent. In addition, the inventor also confirmed that the image enhancement of the T1 contrast agent of the MRI contrast enhancing agent of the present invention can be increased by five folds or more compared to the T1 contrast agent without coupling with the magnetic particle having the T2-reducing effect, and the T2 image enhancement of the magnetic particle can also be increased by two folds or more compared to the magnetic particle alone. Hence, both the T1-reducing effect and the T2-reducing effect (i.e. the sensitivity) for the MRI examination can be improved when the MRI contrast enhancing agent of the present invention is used.

In the MRI contrast enhancing agent of the present invention, the magnetic particle used in the present invention can be any conventional magnetic particle having the T2-reducing effect, and preferably the magnetic particle with both the T2-reducing effect and the T1-reducing effect. The example of the magnetic particle with the T2-reducing effect can be a Fe₃O₄ particle, a FePt particle, a Fe particle, a Mn₃O₄ particle, or a Cd particle. Preferably, the magnetic particle is a Fe₃O₄ particle, which has the T1-reducing effect and the T2*-reducing effect. In addition, the magnetic particle used in the present invention may have a diameter of 1 nm to 10 μm. Herein, the size and the material of the magnetic particle are chosen based on the diagnosing purpose. Preferably, the magnetic particle of the present invention has a diameter of 1 nm to 200 nm. For example, there are several kinds of commercial available Fe₃O₄ particles used in clinic, such as superparamagnetic iron oxide agents (SPIO) with a diameter of about 300 nm to about 4 μm which is generally used through oral administration and for gastrointestinal imaging; standard superparamagnetic iron oxide agents (SSPIO) with a diameter of about 50 nm to about 150 nm which is generally administered through intravenous injection and for organ imaging; and ultrasmall superparamagnetic iron oxide agents (USPIO) with a diameter of about 20 nm to about 40 nm which is also administered through intravenous injection and for blood vessel or lymph node imaging. These commercial available Fe₃O₄ particles can be used in the present invention as the magnetic particle. However, the magnetic particle used in the present invention is not particularly limited to these commercial available Fe₃O₄ particles.

In the MRI contrast enhancing agent of the present invention, the T1 contrast agent can be any conventional T1 contrast agent, such as a gadolinium-based contrast agent. More specifically, the gadolinium-based contrast agent is a Gd-complex formed by a Gd³⁺ ion and a chelating ligand, in which the Gd³⁺ ion coordinates with 7 or 8 coordinating functional groups such as N or COOH in the chelating ligand. The examples of the gadolinium-based contrast agent can be Gd-diethylenetriamine penta-acetic acid (Gd-DTPA), gadobenate dimeglumine (Gd-BOPTA), or gadolinium ethoxybenzyl diethylenetriamine pentaacetic acid (Gd-EOB-DTPA). Preferably, Gd-DTPA is used in the present invention. However, the aforementioned examples of the gadolinium-based contrast agent are only used for illustration, and other gadolinium-based contrast agent or other T1 contrast agents may also be used in the present invention.

In the MRI contrast enhancing agent of the present invention, the T1 contrast agent is conjugated to the magnetic particle through the polymer. Preferably, the polymer used in the present invention is a dendrimer. The examples of the dendrimer used in the present invention comprise a polyethylene glycol (PEG) dendrimer, a polyamidoamine (PAMAM) dendrimer, and a polypropylenimine (PPI) dendrimer. Preferably, the dendrimer used in the present invention is a PEG dendrimer. However, the aforementioned examples of the dendrimer are only used for illustration, and other dendrimer or polymer may also be used in the present invention.

When the magnetic particle and the T1 contrast agent is linked to each other through a polymer and especially a dendrimer, the phagocytosis on the MRI contrast enhancing agent by immune system or liver cell can be inhibited, and therefore the circulation time thereof of the present invention can be prolonged.

The MRI contrast enhancing agent of the present invention comprises the magnetic particle with the T2-reducing effect, the dendrimer coupled to the magnetic particle, and the T1 contrast agent coupled to the dendrimer, so the magnetic particle thereof can enhance the T1-reducing effect of the T1 contrast agent to improve the sensitivity of the MRI contrast enhancing agent, and the dendrimer thereof can further prolong the circulation time thereof. Since the sensitivity and the circulation time thereof are improved, the dose of the MRI contrast enhancing agent can be decreased and the side effect thereof may also be reduced.

In the MRI contrast enhancing agent of the present invention, the molar ratio of the dendrimer conjugated with Fe₃O₄ particles (Fe₃O₄-dendrimer complex) can be in a range from 10: 1 to 200:1. Preferably, the molar ratio of the dendrimer conjugated with Fe₃O₄ particles is in a range from 50:1 to 150:1. More preferably, the molar ratio of the dendrimer conjugated with Fe₃O₄ particles is about 100:1. In the above illustration, Fe₃O₄ particles is one example of the magnetic particles, other magnetic particles such as a FePt particle, a Fe particle, a Mn₃O₄ particle, or a Cd particle can also mixed with the dendrimer in the aforementioned molar ratio.

The molar ratio of the DTPA-Gd conjugation with Fe₃O₄-G3 dendrimer can be in a range from 1:100 to 1:2000. Preferably, the molar ratio of the DTPA-Gd conjugation with Fe₃O₄-dendrimer is in a range from1:300 to 1:1000. In the above illustration, Fe₃O₄ particles and

DTPA-Gd are represented examples for the magnetic particles and the T1 contrast agent respectively, other magnetic particles and T1 contrast agents can also mixed with each other in the aforementioned molar ratio.

In addition, for the diagnosis purpose, the MRI contrast enhancing agent of the present invention may further comprise a targeting molecule coupled to the surface of the magnetic particle or the polymer. The examples of targeting molecule used in the present invention can be any antibodies. For example, when the MRI contrast enhancing agent of the present invention is used to predict or diagnose whether a subject is suffered from lung cancer or not, the targeting molecule used in the present invention can be an anti-EFGR mutation antibody. When the MRI contrast enhancing agent of the present invention is applied to prognosis of head and neck cancers, the targeting molecule used in the present invention can be a specific targeting drug.

When the MRI contrast enhancing agent of the present invention is used to diagnose breast cancer, the targeting molecule can be an anti-Her2 antibody. Here, the present invention only provides some targeting molecule for example, but the present invention is not limited thereto.

The present invention also provides a diagnosing method, which comprises the steps of: administering the aforementioned MRI contrast enhancing agent to a subject; and imaging the subject to generate a MR image. Herein the subject can be a mammal.

Other objects, advantages, and novel features of the invention will become more apparent from the following detailed description when taken in conjunction with the accompanying drawings.

### BRIEF DESCRIPTION OF THE DRAWINGS

FIG. 1A is a perspective view of a MRI contrast enhancing agent according to one preferred embodiment of the present invention;

FIG. 1B is a perspective view of a MRI contrast enhancing agent according to another preferred embodiment of the present invention;

FIG. 2 is a Gd inductively coupled plasma data showing the Gd concentration in mixtures of Fe₃O₄ magnetic particles and Gd-DTPA in different molar ratio; FIG. 3 is a Fe₃O₄ atomic absorption data showing the Fe₃O₄ concentration in mixtures of Fe₃O₄ magnetic particles and Gd-DTPA in different molar ratio;

FIG. 4 is a result of an animal survival test for Fe₃O₄-G3 dendrimer-GdDTPA;

FIG. 5 is a result showing the T1-weighted peak enhancement percentage of Fe₃O₄-G3 dendrimer-GdDTPA by T1-weighted MR images, which is compared with commercially available contrast agent, wherein T1 contrast agent is Gd-DTPA and T2 contrast agent is Resovist; and

FIG. 6 is a result showing the T2-weighted peak enhancement percentage of Fe₃O₄-G3 dendrimer-GdDTPA by T1-weighted MR images, which is compared with commercially available contrast agent, wherein T1 contrast agent is Gd-DTPA and T2 contrast agent is Resovist.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Preparation of Fe₃O₄ Magnetic Particles

To prepare Fe₃O₄ magnetic particles, two-stage additions of protective agent and chemical coprecipitation were employed in the present process. Briefly, 1M of Fe (III) and 2M of Fe (II) aqueous solutions were respectively prepared by dissolving FeCl₂ and FeCl₃. Next, Fe (II) aqueous solution was mixed with Fe (III) aqueous solution in a volume ratio of 1: 4, followed by the addition of organic acid as an adherent. In the present process, 1 ml of Fe (II) aqueous solution was mixed with 4 ml of Fe (III) aqueous solution, and 0.5 g of organic acid was added into the mixture. Then, the pH of the mixture was adjusted with 0.5 M NaOH until the pH of the mixture reached 11 and the color of the mixture was turned into black. The precipitates were collected by a magnet, the supernatant was removed, and the precipitates were washed with deionized water (50 ml in the present process) three times. Excess organic acid (3 g) was added therein to functionalize the surface of the particle with —NH₃⁺ group. The mixture was placed for 5 min and treated with an ultra-sonication for another 30 min. The mixture was added into a solution of water and acetone, and then the precipitates were collected by centrifugation at 7500 rpm for 25 min. The supernatant was removed, and deionized water was added therein to re-suspend the precipitates. After the aforementioned process, Fe₃O₄ magnetic particles were obtained, and the size thereof was determined to be about 126.2±59.4 nm by dynamic light scattering (DLS).

### Preparation of Fe₃O₄ Magnetic Particles Conjugated with Dendrimer

In the present process, polyethylene glycol (PEG) was used as the dendrimer. First, 0.056 g of dendrimer was dissolved in 10 ml of deionized water. 0.1 g of succinic anhydride was dissolved in 5 ml of deionized water in dark. 1 ml of succinic anhydride solution was added into 10 ml of dendrimer solution. The mixture was inversed to mix completely, placed in dark and reacted overnight at room temperature. After the aforementioned process, PEG-OH₅—COOH₄ (hereafter, also called G3 dendrimer) was obtained.

The obtained G3 dendrimer was resuspended into 10 ml of deionized water, and the final concentration of the G3 dendrimer solution was about 1786 μm. 10 mg/ml of Fe₃O₄ magnetic particles was mixed with G3 dendrimer solution in a molar ratio of G3 dendrimer conjugated with Fe₃O₄ magnetic particles being 100:1, 0.01 g of EDC was added into the mixture, and then the mixture was mixed and reacted at 4° C. overnight. The precipitants were collected by centrifugation at 13000 rpm for 30 min, and then the supernatant was removed. 1 ml of deionized water was further added therein, the precipitants were again collected by centrifugation at 13000 rpm for 5 min, and then the supernatant was removed. Finally, the precipitants were resuspended with 100 μl of deionized water, and the Fe₃O₄ magnetic particles with G3 dendrimer coupled thereon were obtained. After DLS determination, the size thereof was determined to be about 144.1±60 nm, which means the G3 dendrimer were successfully coupled to surfaces of the Fe₃O₄ magnetic particles.

### Preparation of Fe₃O₄-G3 Dendrimer-DTPA-GdCl₃

Here, DTPA was used as a chelating ligand for Gd³⁺.

First, 0.035732 g of DTPA anhydride was dissolved in DMSO (10 ml), and placed at 60° C.-70° C. overnight. Next, Fe₃O₄ magnetic particles coupled with G3 dendrimer (hereafter, also called Fe₃O₄-G3 dendrimer) was mixed with DTPA of 1:1000. Here, 100 μl of Fe₃O₄-G3 dendrimer (25 μM) was mixed with 0.2 ml of DTPA anhydride to obtain Fe₃O₄-G3 dendrimer-DTPA. 0.067 g of GdCl₃ was dissolved in 5 ml of 1N NaOH. Then, Fe₃O₄-G3 dendrimer-DTPA and GdCl₃ were mixed in a molar ratio of 1:1000, 1:700, 1:500 or 1:300.

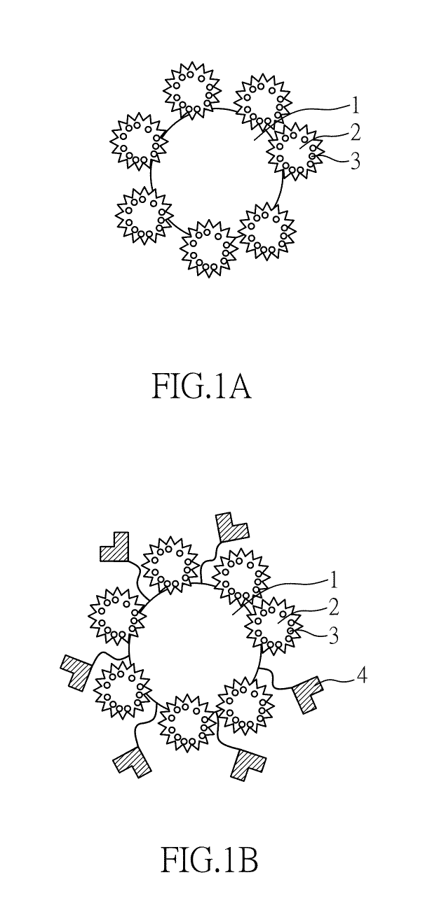

After the aforementioned process, Fe₃O₄-G3 dendrimer-GdDTPA as a MRI contrast enhancing agent were obtained, and the perspective view thereof was shown in FIG. 1. As shown in FIG. 1A, the MRI contrast enhancing agent comprises: a Fe₃O₄ magnetic particle 1 with a T2-reducing effect; a G3 dendrimer 2 as a polymer coupled to a surface of the Fe₃O₄ magnetic particle; and a Gd-complex 3 as a T1 contrast agent, which is formed by a Gd³⁺ ion and a chelating ligand (DTPA) and coupled to the G3 dendrimer 2.

### Preparation of Fe₃O₄-G3 Dendrimer-GdDTPA with Targeting Molecules (Fe₃O₄-G3 Dendrimer-GdDTPA-EG2)

Here, EG2 SdAb obtained from National Research Council (NRC) was used as a targeting molecule.

One milliliter of 0.1 μM (particle concentration) Fe₃O₄ with —NH₃ modification was added to 1 ml of 0.2M Nα, Nα-Bis (carboxymethyl)-L-lysine (NTA) and 400 μl of 55% (w/w) gultaraldehyde at room temperature for stirring for 8 h. After conjugation Fe₃O₄ nanoparticles with NTA, 2 ml of 1.0M NiSO₄ was added and the mixture was stirred at room temperature for different time periods. When the incubation was completed, the mixture was centrifuged at 13000 rpm for 5 min and the supernatant was removed. The resulting precipitates (Fe₃O₄₋0 NiNTA-G3 dendrimer-GdDTPA) were redispersed in 2 ml H₂O and centrifuged at 13000 rpm for 5 min to remove the excess Ni²⁺ ions. Finally, EG2 will self-assembly to Fe₃O₄₋ NiNTA-G3 by moral ratio 10:1.

As shown in FIG. 1B, the MRI contrast enhancing agent may further comprise: a targeting molecule 4 coupled to the surface of the magnetic particle 1, or the polymer 2.

### Evaluation the Effect of the Prepared Fe₃O₄-G3 Dendrimer-GdDTPA

In order to understand the binding affinity between Fe₃O₄ magnetic particles and Gd-DTPA, different molar ratio of Fe₃O₄ magnetic particles and Gd-DTPA were mixed according to Table 1.

TABLE 1

| | Molar ratio |
|---|---|
| Group number | Fe₃O₄-G3 dendrimer:Gd-DTPA |
| 1 | 1:300 |
| 2 | 1:500 |
| 3 | 1:700 |
| 4 | 1:1000 |

The obtained mixtures were examined with Inductively Coupled Plasma. A know weight of the Fe₃O₄-G3 dendrimer-GdDTPA nanoparticles was dissolving in deionized water in serial dilution and the aqua regia was added followed by a drop, since the Fe₃O₄-G3 dendrimer-GdDTPA nanoparticles will be digested original structure by ague regia and released Gd and Fe ions in solution. The solution contents of Gd and Fe ions were analyzed by inductively coupled plasma-atomic emission spectrometer (ICP-ASE).

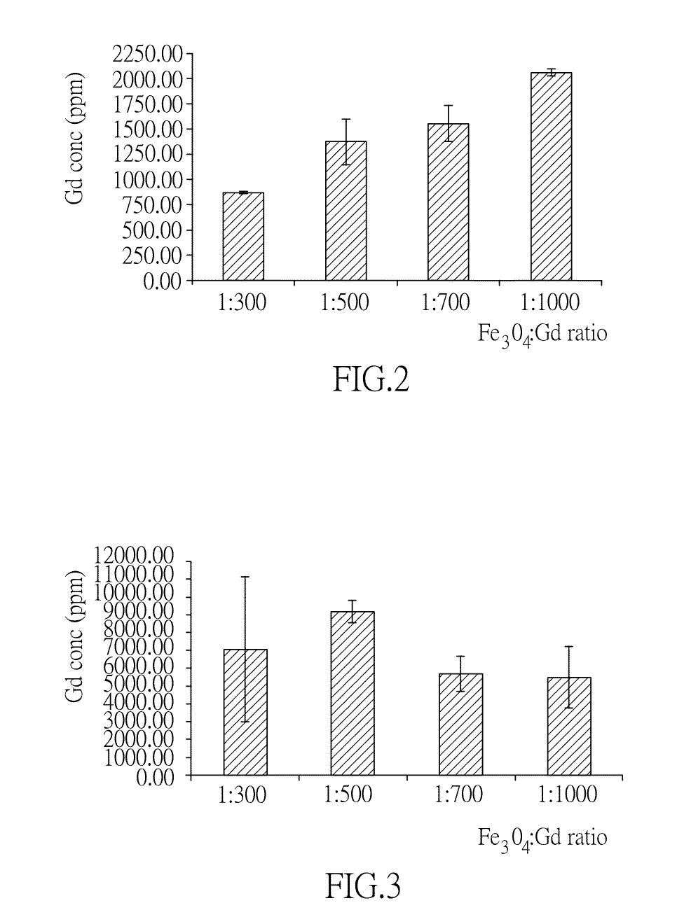

After 1000 folds dilution the mixture, the Fe₃O₄-G3 and GdDTPA was mixed in 1:300, 1:500, 1:700, and 1:1000 molar ratio. As shown in FIG. 2, the values of the Gd concentration (Gd conc) in ICP increased depending on the increase in the proportion of Fe₃O₄-G3:GdDTPA (Fe₃O₄:Gd ratio), and the result of Gd concentration in Fe₃O₄-G3 dendrimer-GdDTPA nanoparticles, as expected, was the increase from about1000 ppm to 2000 ppm as the ratio of Fe₃O₄:Gd increased. In addition, as shown in FIG. 3, after 1000 folds dilution of the mixture (Fe₃O₄-G3 dendrimer-GdDTPA nanoparticles), the detected Fe₃O₄ concentration (Fe conc) in each group was about 7000-9000 ppm. This result indicates that the amount of Gd³⁺ coupled to Fe₃O₄ magnetic particles is related to the added amount of Gd³⁺.

Here, the survival test of Fe₃O₄-G3 dendrimer-GdDTPA (1:1000) will compare with the same concentration of Gd-DTPA. The survival test were determined in vivo using 6-week-old BALB/c mice (n=12). The contrast agents were injected into the tail vein of each mouse. The survival and pathological symptoms and signs of each mouse were monitored for a period of one month.

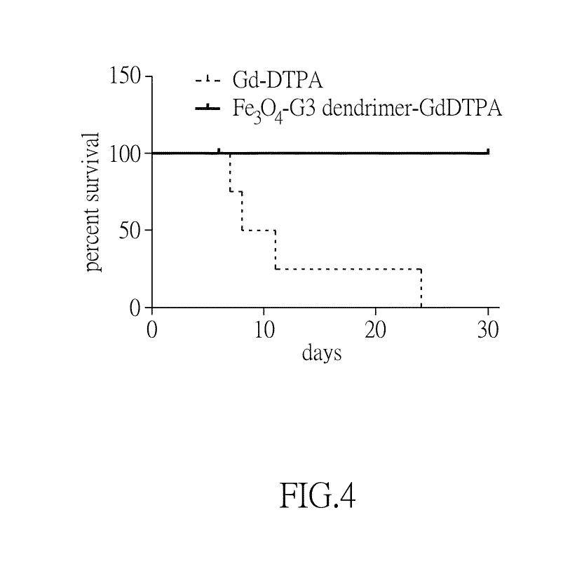

As shown in FIG. 4B, the Fe₃O₄-G3 dendrimer-GdDTPA more biocompatibility than Gd-DTPA.

Hereafter, the MRI contrast enhancing agent prepared in the present embodiments are evaluated as follows.

All MR imaging measurements were performed with a 9T systems. T1-weighted MR images were acquired using a conventional spin-echo sequence under the following parameters: TR/TE=783/17ms, 835×1671 matrices , 180×180mm field of view, 6.41 Hz/Px of bandwidth, a slice thickness of 2 mm. T2-weighted MR image using a fast spin-echo sequence was used to reduce acquisition time under the following parameters: TR/TE=765/12ms, 256×256 matrices, 180×180mm field of view and 6.41 Hz/Px of bandwidth, a slice thickness of 2 mm.

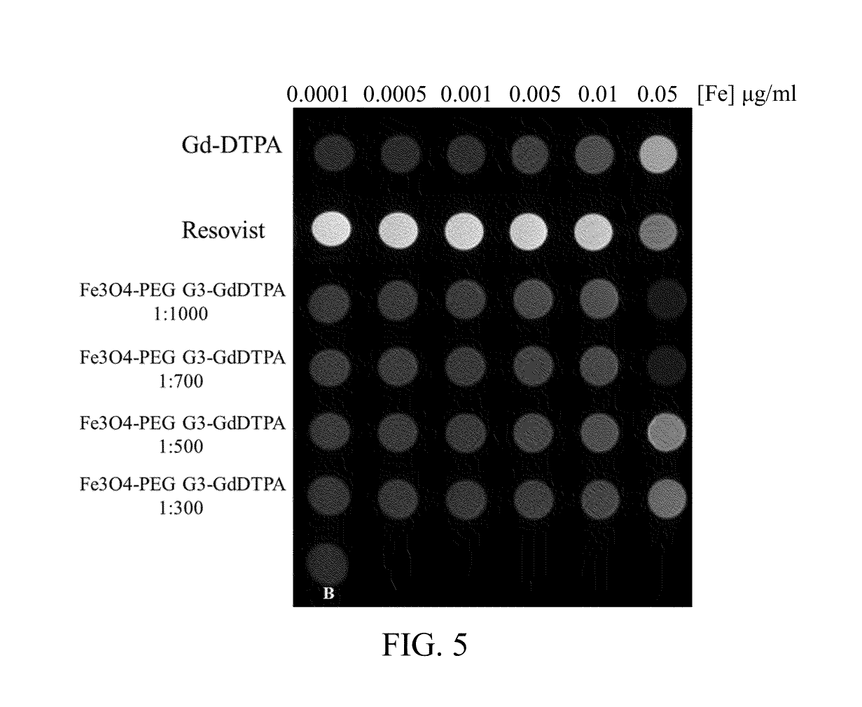

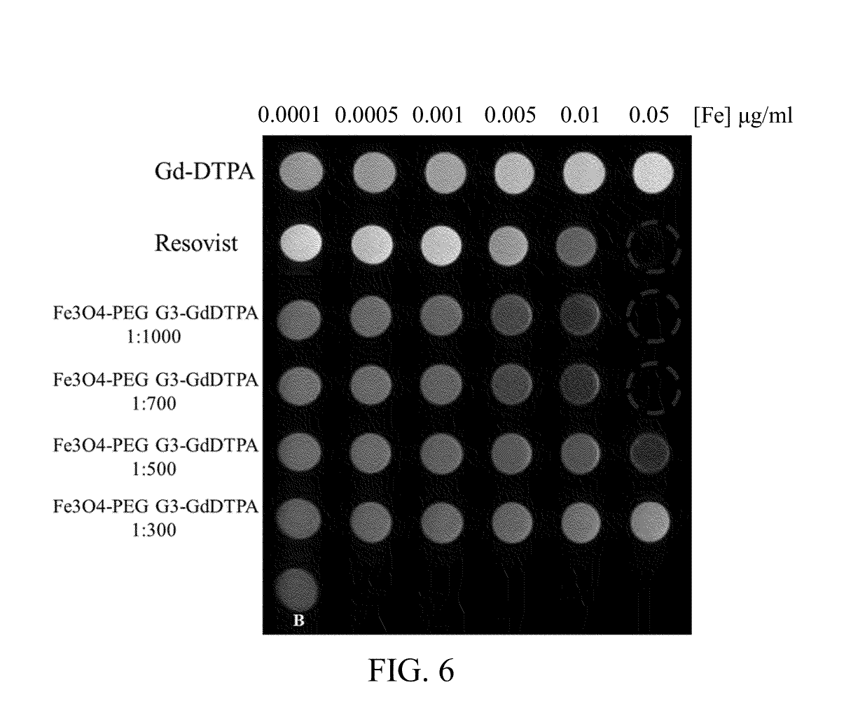

FIG. 5 and FIG. 6 respectively show the T1- and T2-weighted images 0.0001, 0.0005, 0.001, 0.005, 0.01, 0.05 μg/ml individually in different molar ratio injection of Fe₃O₄-G3 dendrimer-GdDTPA, respectively. Compared with the image without particle injection which is indicated by “B” in FIG. 5 and FIG. 6, T1 and T2 weighted MR images of 0.0001, 0.0005, 0.001, 0.005, 0.01 μg/ml post injection did not show obvious changes. As shown in FIG. 5, the T1-weighted MR images using the MRI contrast enhancing agents of the present embodiment were brighter than those using GdDTPA alone. As shown in FIG. 6, the T2-weighted MR images using the MRI contrast enhancing agents of the present embodiments were darker than those using commercial Resovist alone. These results demonstrated that Fe₃O₄-PEG-G3 dendrimer-GdDTPA nanoparticles (NPs) owing high r1 and r2 reaxivities exhibited both positive T1 and negative T2 contrast enhancement MR images. The developed multifunctional Fe₃O₄-PEG-G3 dendrimer-GdDTPA NPs are able to provide not only specific targeting effect but also dual MR imaging. The T1 and T2-weighted MR images show that the Fe₃O₄-G3 dendrimer-GdDTPA conjugated with EG-2 was successfully developed as a tumor-targeting carrier to deliver the contrast agents for the identifiable diagnosis of tumor (data not show). In addition, the T1 and T2-weighted MR images show that the Fe₃O₄-G3 dendrimer-GdDTPA used as a dual T1 and T2 contrast agent for MR imaging.

Although the present invention has been explained in relation to its preferred embodiment, it is to be understood that many other possible modifications and variations can be made without departing from the spirit and scope of the invention as hereinafter claimed.

1. A MRI contrast enhancing agent, comprising:
a magnetic particle with a T2-reducing effect;
a polyethylene glycol (PEG) dendrimer coupled to a surface of the magnetic particle; and
a T1 contrast agent coupled to the polyethylene glycol (PEG) dendrimer.

2. The MRI contrast enhancing agent as claimed in claim 1, wherein the magnetic particle has both the T2-reducing effect and a T1-reducing effect.

3. The MRI contrast enhancing agent as claimed in claim 1, wherein the magnetic particle is a Fe₃O₄ particle, a FePt particle, a Fe particle, a Mn₃O₄ particle, or a Cd particle.

4. The MRI contrast enhancing agent as claimed in claim 3, wherein the magnetic particle is a Fe₃O₄ particle.

5. The MRI contrast enhancing agent as claimed in claim 1, wherein the T1 contrast agent is a gadolinium-based contrast agent.

6. The MRI contrast enhancing agent as claimed in claim 5, wherein the gadolinium-based contrast agent is a Gd-complex formed by a Gd³⁺ ion and a chelating ligand.

7. The MRI contrast enhancing agent as claimed in claim 6, wherein the gadolinium-based contrast agent is Gd-DTPA, Gd-BOPTA, or Gd-EOB-DTPA.

8-10. (canceled)

11. The MRI contrast enhancing agent as claimed in claim 1, further comprising a targeting molecule coupled to the surface of the magnetic particle, or the polyethylene glycol (PEG) dendrimer.

12. The MRI contrast enhancing agent as claimed in claim 1, wherein the magnetic particle has a diameter of 1 nm to 10 μm.

13. The MRI contrast enhancing agent as claimed in claim 12, wherein the magnetic particle has a diameter of 1 nm to 200 nm.

14. The MRI contrast enhancing agent as claimed in claim 1, wherein the polyethylene glycol (PEG) dendrimer is coupled to the magnetic particle in a molar ratio of 10:1 to 200:1 to form a magnetic particle-dendrimer complex.

15. The MRI contrast enhancing agent as claimed in claim 14, wherein the T1 contrast agent is coupled to the magnetic particle-dendrimer complex in a molar ratio of 1:100 to 1:2000.
